SEQUENCE LISTING (67 sequences)

SEQ ID NO: 1 - DNA, 25 bp, Cyanobacteria
aaaagtgttttattagcggctcatt

SEQ ID NO: 2 - DNA, 26 bp, Cyanobacteria
tctaaccgtccattagagactaaacc

SEQ ID NO: 3 - DNA, 24 bp, Cyanobacteria
ctgcacacaacaccatctatttac

SEQ ID NO: 4 - DNA, 22 bp, Cyanobacteria
gatcgcagtttctcggtctaat

SEQ ID NO: 5 - DNA, 25 bp, Cyanobacteria
gctccaggtgtgattgaatttatag

SEQ ID NO: 6 - DNA, 23 bp, Cyanobacteria
gcatagcggctaatttggtattc

SEQ ID NO: 7 - DNA, 23 bp, Cyanobacteria
cccatctaccacgatgtctttac

SEQ ID NO: 8 - DNA, 22 bp, Cyanobacteria
cactgcatggctattgactact

SEQ ID NO: 9 - DNA, 18 bp, Cyanobacteria
tgcgaactgctgccataa

SEQ ID NO: 10 - DNA, 20 bp, Cyanobacteria
cagcaaatgcaacacggaat

SEQ ID NO: 11 - DNA, 27 bp, Cyanobacteria
ctgaataatcagagggatattgttacg

SEQ ID NO: 12 - DNA, 24 bp, Cyanobacteria
ctccagataactctaaacgtaggg

SEQ ID NO: 13 - DNA, 22 bp, Cyanobacteria
ctgctcaacccttaagtctagg

SEQ ID NO: 14 - DNA, 20 bp, Cyanobacteria
cgactaatacggcggctaaa

SEQ ID NO: 15 - DNA, 20 bp, Cyanobacteria
ttacagctaacgggtggaac

SEQ ID NO: 16 - DNA, 22 bp, Cyanobacteria
gtaactccgctaagggataacg

SEQ ID NO: 17 - DNA, 21 bp, Cyanobacteria
atggccctattcaaaggtcag

SEQ ID NO: 18 - DNA, 19 bp, Cyanobacteria
ccatctgcgcaaacaacag

SEQ ID NO: 19 - DNA, 22 bp, Cyanobacteria
cactcaatgaaaccgggaaatc

SEQ ID NO: 20 - DNA, 21 bp, Cyanobacteria
ccgatgggatgtttggttaga

SEQ ID NO: 21 - DNA, 23 bp, Cyanobacteria
tgctggctattacaacctctatg

SEQ ID NO: 22 - DNA, 21 bp, Cyanobacteria
caggcttgtccaataggaact

SEQ ID NO: 23 - DNA, 22 bp, Cyanobacteria
gcgggactttatgctctactac

SEQ ID NO: 24 - DNA, 20 bp, Cyanobacteria
tactccgtcatcggcatttg

SEQ ID NO: 25 - DNA, 21 bp, Cyanobacteria
cgctatacccacggatttgtt

SEQ ID NO: 26 - DNA, 22 bp, Cyanobacteria
gggatcagcagtagtccatcta

SEQ ID NO: 27 - DNA, 21 bp, Cyanobacteria
aacacggctttgaggtctatc

SEQ ID NO: 28 - DNA, 22 bp, Cyanobacteria
gaagtaatcctcacaggttccc

SEQ ID NO: 29 - DNA, 28 bp, Cyanobacteria
aggtcaatggctatcgtgtagatccagg

SEQ ID NO: 30 - DNA, 24 bp, Cyanobacteria
taacactgctgtcaccgagttgcc

SEQ ID NO: 31 - DNA, 24 bp, Cyanobacteria
taaagcctggaggttgagagtggc

SEQ ID NO: 32 - DNA, 24 bp, Cyanobacteria
taatattgcggcagaaacggctgc

SEQ ID NO: 33 - DNA, 24 bp, Cyanobacteria
aatggacggttagaagcagccgat

SEQ ID NO: 34 - DNA, 28 bp, Cyanobacteria
tgagttatggagttgaagaaagcctcga

SEQ ID NO: 35 - DNA, 24 bp, Cyanobacteria
ttgcaacggaacgggatagtctgg

SEQ ID NO: 36 - DNA, 24 bp, Cyanobacteria
cactgcgttccatttctgcgatgc

SEQ ID NO: 37 - DNA, 26 bp, Cyanobacteria
cccaagtaaattatgttgcaccgcgt

SEQ ID NO: 38 - DNA, 24 bp, Cyanobacteria
taccgagtctcaccgcccgatatt

SEQ ID NO: 39 - DNA, 24 bp, Cyanobacteria
attgagagcgtggtgggtgtaact

SEQ ID NO: 40 - DNA, 22 bp, Cyanobacteria
caaagagaggtggaagaggaag

SEQ ID NO: 41 - DNA, 22 bp, Cyanobacteria
gggtctactccatactcctcaa

SEQ ID NO: 42 - DNA, 25 bp, Cyanobacteria
ttgaatgtgagtacccaagaggccg

SEQ ID NO: 43 - DNA, 24 bp, Cyanobacteria
gctaacctcactaacgaactactc

SEQ ID NO: 44 - DNA, 22 bp, Cyanobacteria
gagaacattcacacgctctact

SEQ ID NO: 45 - DNA, 28 bp, Cyanobacteria
caacactgctgtcactgaattaccctct

SEQ ID NO: 46 - DNA, 23 bp, Cyanobacteria
tggaatgttaccagcaggaataa

SEQ ID NO: 47 - DNA, 20 bp, Cyanobacteria
agtgctgcttgacgtaaact

SEQ ID NO: 48 - DNA, 22 bp, Cyanobacteria
atgcctatccgtgaaggtaaag

SEQ ID NO: 49 - DNA, 20 bp, Cyanobacteria
ccaccggagtgagcatattt

SEQ ID NO: 50 - DNA, 21 bp, Cyanobacteria
atcgttcaacacgcagaattg

SEQ ID NO: 51 - DNA, 22 bp, Cyanobacteria
tcatccatttcgataggtgtgg

SEQ ID NO: 52 - DNA, 95 bp, Cyanobacteria
aaaagtgttttattagcggctcattttcgggtattaagtttactgaataatcagagggatattgttacaggtttagtctctaatggacggttaga

SEQ ID NO: 53 - DNA, 105 bp, Cyanobacteria
gctccaggtgtgattgaatttatagggcgaaaagataatcaagttaaggtcaatggctatcgtgtagatccaggagaaattgaataccaaattagccgctatgcc

SEQ ID NO: 54 - DNA, 120 bp, Cyanobacteria
ctgcacacaacaccatctatttaccgagaaattttagctgtactcgcacccgaagaaactatccccagcttaaaatatatctcttgcgggggagaaaaattagaccgagaaactgcgatc

SEQ ID NO: 55 - DNA, 99 bp, Cyanobacteria
cccatctaccacgatgtctttacttaccatattcgagccactctcaacctccaggctttacacagtgccattaaacaagtagtcaatagccatgcagtg

SEQ ID NO: 56 - DNA, 142 bp, Cyanobacteria
tgcgaactgctgccataattgcttctgtkccmgtattrctaaaagcmacccgytcyacaccagttatttcactaaytaaagcagccgtttctgccgcaatattagattgcattcctaatcckattccgtgttgcatttgctg

SEQ ID NO: 57 - DNA, 124 bp, Cyanobacteria
ctgaataatcagagggatattgttacgggtttagtctctaatggacggttagaagcagccgatggggaaaagatattaggtttatttttgaatactttgcccctacgtttagagttatctggag

SEQ ID NO: 58 - DNA, 129 bp, Cyanobacteria
ctgctcaacccttaagtctaggcacccctttaggaatggttgaagacgtaatagtcttgagttatggagttgaagaaagcctcgatattattgctactcatgctgatgatttagccgccgtattagtcg

SEQ ID NO: 59 - DNA, 100 bp, Cyanobacteria
ttacagctaacgggtggaacctggttagacttagtacgacaagtttttgcaacggaacgggatagtctggcctggcgacgttatcccttagcggagttac

SEQ ID NO: 60 - DNA, 102 bp, Cyanobacteria
atggccctattcaaaggtcaggccagctggcgttccatgagccaggaattgaagccactgcgttccatttctgcgatgcagtcctgttgtttgcgcagatgg

SEQ ID NO: 61 - DNA, 112 bp, Cyanobacteria
cactcaatgaaaccgggaaatctacccaagtaaattatgttgcaccgcgtaataatttagagtcaaacctagttagaatctgggaaaagattctaaccaaacatcccatcgg

SEQ ID NO: 62 - DNA, 185 bp, Cyanobacteria
caaagagaggtggaagaggaaggtgaaaattctaactgtgtgcttgtagttgagcgtttcttgaatgtgagtacccaagaggccgctaacctcactaacgaactactcaactcccgtttataccaatttgacaacactgctgtcactgaattaccctctctttttgaggagtacggagtagatcc

SEQ ID NO: 63 - DNA, 123 bp, Cyanobacteria
gctaacctcactaacgaactactcaactcccgtttataccaatttgacaacactgctgtcactgaattaccctctctttttgaggagtacggagtagatccagtagagcgtgtgaatgttctc

SEQ ID NO: 64 - DNA, 109 bp, Cyanobacteria
tgctggctattacaacctctatggtccgacggagacaaatgtctgcacatattaccgagtctcaccgcccgatattgaaacaagtgaagcagttcctattggacaagcc

SEQ ID NO: 65 - DNA, 111 bp, Cyanobacteria
gcgggactttatgctctactactgtaccctgaaaggcggcattgagagcgtggtgggtgtaactcgctgtcgcaattatgtcaattattcccaaatgccgatgacggagta

SEQ ID NO: 66 - DNA, 395 bp, Cyanobacteria
cgctatacccacggatttgttgcgattccagtgatcctcgcttgtcgagaaaaaggttttttcgagctactagcccatgaaagtcctctctccttagagcaaatggtcaagcatctgggagctaataccggacatttccaagttgctttgaggatgctcgaatctttacattggctttcccgaaatgagcaacttaaatattctctgacctcagaagcagcgattcacaaccaaattccagaagacgttctcgagttgtaccacctaccaattgagtcttatttacaaggaaaacaagaaaagttgctgggaagatggattgatcgttcttgccaactgtggaatctggataatcccttaatagcagattttttagatggactactgctgatccc

SEQ ID NO: 67 - DNA, 113 bp, Cyanobacteria
aacacggctttgaggtctatccaattccctttcgcaatgtctttgagtttggcggttcgctccattgtgccacctgggatatccatcgcacgggaacctgtgaggattacttc

(19) United States
(12) Patent Application Publication
(10) Pub. No.: US 2024/0191311 A1
(43) Pub. Date: Jun. 13, 2024

(54) EARLY WARNING GENETIC TESTING OF TOXIC CYANOBACTERIA IN WATER SUPPLY

(71) Applicant: USA, represented by Administrator of the U.S. EPA, Washington, DC (US)

(72) Inventor: Jingrang Lu, Mason, OH (US)

(73) Assignee: The United States Government, as represented by the Administrator of the U.S. EPA, Washington, DC (US)

(21) Appl. No.: 16/142,319

(22) Filed: Sep. 26, 2018

(51) Int. Cl. C12Q 1/689; C12Q 1/686

(52) U.S. Cl. CPC C12Q 1/689; C12Q 1/686; C12Q 2600/142

(57) ABSTRACT

This invention is at least one panel of qPCR/RT-qPCR assays which enables simultaneous testing for the presence of multiple species and subgroups of cyanobacteria that produce microcystin, anatoxin, saxitoxin, and cylindrospermopsin cyanotoxins. The method takes into account that some cyanobacteria species may carry genes associated with multiple toxin types. Testing for each toxin type is conducted under standardized test conditions which allow quantification of the number of gene copies present for cyanotoxins which may contribute to the overall toxin level.

### STATEMENT REGARDING FEDERALLY SPONSORED RESEARCH OR DEVELOPMENT

The invention described herein was made by an employee of the United States Government and may be manufactured and used by the Government of the United States of America for governmental purposes without the payment of any royalties. This and related patents are available for licensing to qualified licensees. Please contact Carmen Krieger at 202.564.0396 for more information.

### INCORPORATION OF SEQUENCE LISTING

Incorporated by reference in its entirety herein is a computer-readable nucleotide sequence listing submitted concurrently herewith and identified as follows: One 12,418 Byte ASCII (Text) file named “EPA_800-17_SEQ.txt,” created on Aug. 29, 2018.

### FIELD OF INVENTION

The present invention relates to nucleic acid products used in the analysis of nucleic acids, such as primers or probes for detection or identification of organisms for bacteria, and more specifically to an assay for simultaneously conducting testing for a plurality of cyanobacteria which carry a gene to produce a toxin, using standardized test conditions.

### BACKGROUND OF THE INVENTION

The U.S. Environmental Protection Agency (EPA) publishes an annual list of the top thirty unregulated contaminants that are known or expected to occur in public water systems in the U.S. Ten of the thirty contaminants of concern are toxins produced by a common type of bacteria called cyanobacteria.

Cyanobacteria, also called blue-green algae, are microscopic organisms found naturally in all types of water. A “cyanobacterial bloom” is an event during which cyanobacteria, multiply very quickly. Blooms can form in warm, slow-moving waters that are rich in nutrients from fertilizer runoff or septic tank overflows, and most often occur in late summer or early fall.

A harmful cyanobacterial bloom is an event associated with elevated cyanotoxin levels that are either deemed unsafe or require further monitoring. Exposure to high cyanotoxin levels causes damage to liver cells and neural signaling pathways in humans, as well as less severe effects such as skin rashes. These events occur on a global basis at great risk to local populations. For example, in 2014, a toxic bloom left 500,000 people in Ohio without drinking water. In 2014, a toxic bloom caused Florida to declare a state of emergency.

Most cyanobacterial blooms do not produce toxins at a sufficient level to compromise public water supplies and cause harm to humans and other species. Additionally, the vast majority of cyanobacteria species do not carry the gene necessary to produce toxins. However, several types of cyanobacteria carry genes which produce one or more types of toxins during a bloom. The aggregate level of all types of cyanotoxins produced by all species known to be carriers may cause the toxin level to exceed a safe threshold for humans and other species.

Historically, water supplies have been monitored by measuring cyanobacteria count and biomass to determine the presence of cyanobacterial species and their blooms, without differentiating species that carry harmful toxin genes or the types of toxins produced.

More recently, assays have been developed to test for genes associated with microcystin, anatoxin, saxitoxin and cylindrospermopsin by performing quantitative polymerase chain reaction (qPCR) and reverse transcription qPCR (RT-qPCR) methods known in the art. These test methods known in the art can detect the presence of a single toxin gene type produced by multiple species.

RT-qPCR and qPCR testing must be performed under different time and temperature conditions for each toxin type, and testing methods must be further differentiated for individual species. The number of gene copies detected can be correlated to future levels for the individual toxin. However, the test conditions of each assay used to test an individual toxin gene are not uniform, and the quantifications produced using each assay are not statistically comparable. Therefore, the results cannot be aggregated to predict a total toxin level.

There is an unmet need for a single assay kit which can simultaneously test for multiple common types of cyanotoxin producing genes across diverse species. There is a further unmet need for testing methods that can be used to accurately determine the probability that a cyanobacteria bloom will result in cyanotoxin levels that exceed US EPA 10-day health advisory levels for drinking water.

### SUMMARY OF THE INVENTION

This invention is a system comprised of quantitative polymerase chain reaction (qPCR) and reverse transcription qPCR (RT-qPCR) assays for detecting the presence of cyanotoxin genes. In various embodiments, the invention includes one or two panels of assays for screening and early warning and for toxic group identification.

The invention enables simultaneous testing for the presence of cyanobacteria genes associated with four toxins which contribute to the cyanotoxin level in a water supply, and the presence of dominant toxic cyanobacterial groups which may trigger the need for an EPA Health Advisory alert. The toxin types associated with the tested genes are anatoxin, microcystin, saxitoxin and cylindrospermopsin. The invention includes qPCR assay panels comprised of separate assays to simultaneously detect each toxin type and toxic group. The assays operate under standardized test conditions. The standardized test conditions include a uniform annealing temperature, thermocycle duration and control samples having common parameters. Results obtained using these standardized conditions can be used to determine the aggregate number of gene copies present in a sample for the four toxin types tested.

In various embodiments, the assay contained in each assay panel has a common annealing temperature of approximately 62 degrees Celsius.

In various embodiments, the method of use includes steps for producing mathematically comparable test results for each toxin quantifying the number of cyanotoxin gene copies detected by each assay and producing test data which may be aggregated for multiple toxin types.

### TERMS OF ART

As used herein, the term, “aggregate number of gene copies” means the total number of gene copies present in a sample for the four toxins tested which contribute to overall toxin levels.

As used herein, the term, “comparable test results” means test data which is obtained under standardized test conditions so that it is mathematically comparable and may be aggregated and analyzed relative to multiple toxin types.

As used herein, the term, “standardized test conditions” means a set of common parameters for multiple qPCR/RT-qPCR tests which results in comparable test results.

### BRIEF DESCRIPTION OF THE DRAWINGS

FIG. 1 is a diagram which illustrates how a Cyanotoxin Prediction (CTP) Assay Panel can be used to more accurately detect the presence of toxin-producing cyanobacteria in a water sample.

FIG. 2 is a table identifying multiple cyanotoxin types, the carrier group of cyanobacteria associated with each cyanotoxin gene, and the common DNA sequences which define a member of the cyanobacteria carrier group.

FIG. 3 is a table illustrating exemplary primer and probe sequences which can be used to produce a CTP Assay Panel for early detection and warning.

FIG. 4 is a table illustrating primer and probe sequences which can be used to produce a CTP Assay Panel for detecting dominant toxic groups of cyanobacteria.

FIG. 5 illustrates the common genetic sequences which are detected by the primers and probes used to produce a CTP Assay Panel for detecting dominant toxic groups of cyanobacteria.

FIG. 6 illustrates an exemplary method of using a CTP Assay Panel and correlating CTP Assay Panel results to subsequently measured cyanotoxin levels.

FIG. 7 summarizes exemplary data reporting the number of toxic gene copies and the concentration of cyanotoxins measured during periodic testing of a water source.

FIG. 8 summarizes exemplary data correlating the number of toxic gene copies measured by the CTP Assay Panel to the subsequent concentration of cyanotoxins measured by an enzyme-linked immunosorbent assay (ELISA).

### DETAILED DESCRIPTION OF THE INVENTION

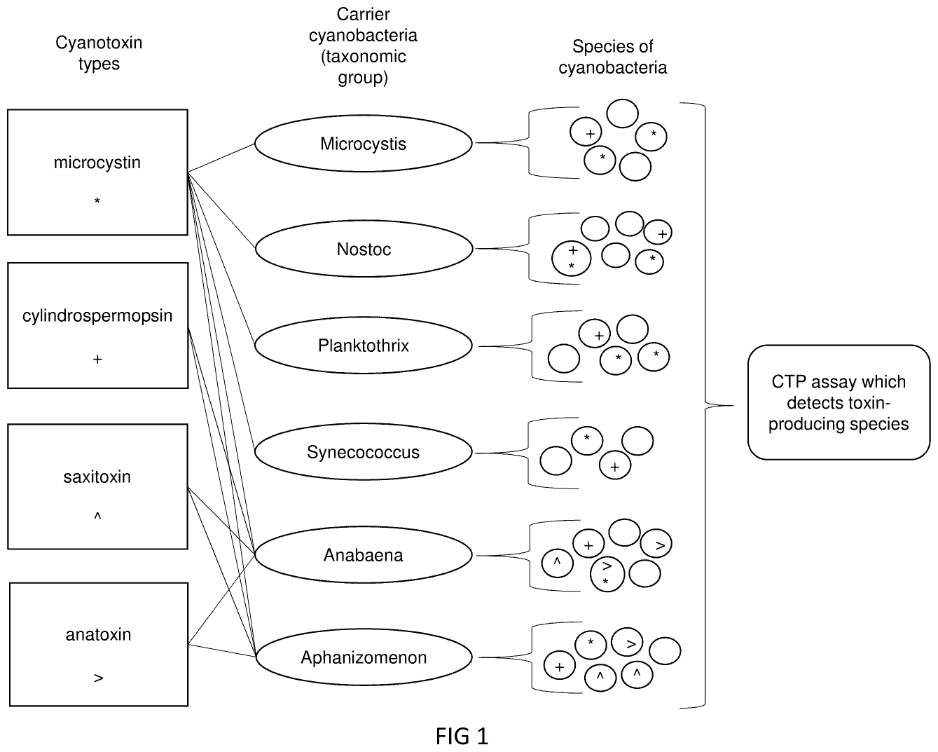

FIG. 1 is a diagram which illustrates how one exemplary embodiment of a Cyanotoxin Prediction (CTP) Assay Panel can be used to more accurately detect the presence of toxin-producing cyanobacteria in a water sample.

FIG. 1 illustrates four known types of cyanotoxins (microcystin, anatoxin, saxitoxin, and cylindrospermopsin) which are produced by more than one subgroup of cyanobacteria, represented as ovals. Each subgroup of cyanobacteria includes multiple species, represented as circles; however, only a few of these species produce cyanotoxins. Species carrying toxic genes that produce cyanotoxins are represented by an asterisk or symbol in the circle.

In the exemplary embodiment shown, the CTP Assay Panel distinguishes between toxic and non-toxic species to specifically detect the presence of toxic species.

The CTP Assay Panel identifies and distinguishes the presence of toxic subgroups of cyanobacteria through the use of novel oligonucleotide primers and quantitative polymerase chain reaction (qPCR) amplification methods known in the art.

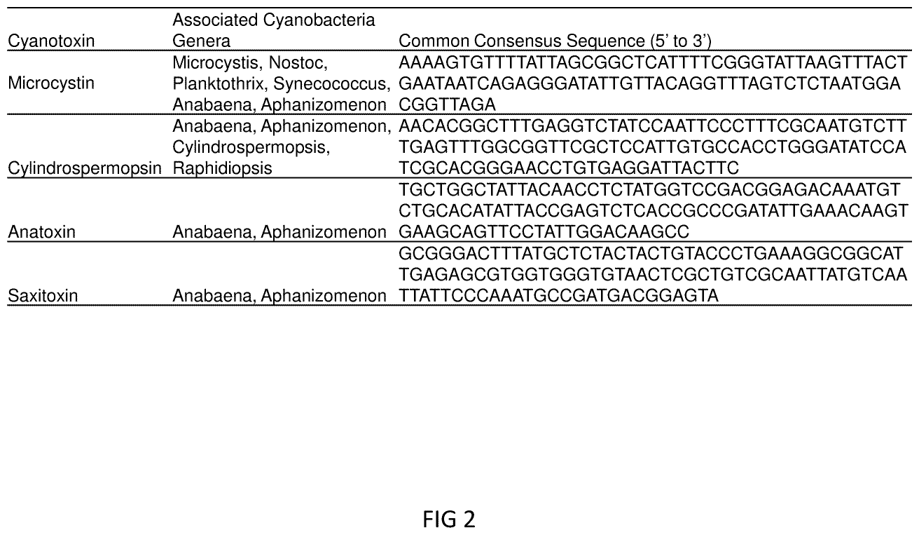

FIG. 2 is a table identifying multiple cyanotoxins, the carrier group of cyanobacteria associated with each cyanotoxin gene, and the common DNA sequences which define a member of the cyanobacteria carrier group.

The right-most column illustrates the common DNA sequences identified by the invention. These sequences, also called consensus sequences, are common in multiple species and allow simultaneous testing for four different toxin genes to simultaneously detect the presence of multiple species that produce cyanotoxins.

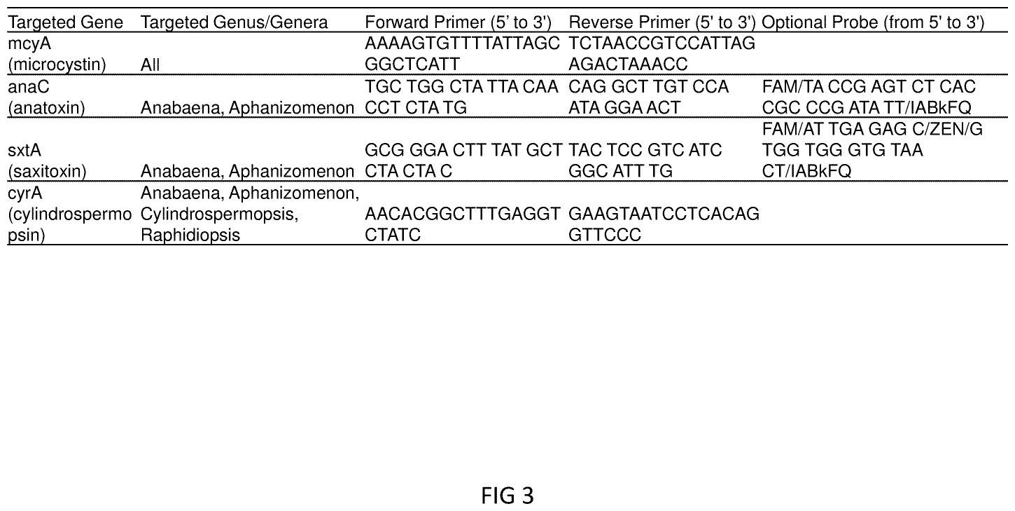

FIG. 3 is a table illustrating exemplary primer and probe sequences which can be used to produce CTP Assay Panel 100 for early detection and warning.

In one exemplary embodiment, CTP Assay Panel 100 is a panel of RT-qPCR/qPCR assays for detecting cyanotoxin genes, which include the novel primer pairs described in FIG. 3. In this embodiment, the primer pairs are designed to detect multiple species of toxic cyanobacteria simultaneously. The primers shown each have a sequence that will bind to a cyanotoxin gene at 60-64 degrees Celsius. In various embodiments, the recommended annealing temperature for the primers shown is 62 degrees Celsius. This common annealing temperature allows all assays to be conducted simultaneously. Without these novel primers and standardized conditions for multiple species, it was not possible to integrate all of the test results for multiple species. Standardized qPCR reaction conditions produce statistically comparable qPCR data from samples with different species, taken from geographically diverse waters.

In this exemplary embodiment, the assays are standardized with the same common annealing temperature, thermocycle duration, and control samples designed to yield consistent qPCR test results. In various embodiments, CTP Assay Panel 100 further includes approximately four to six positive control samples, each having a unique number of cyanotoxin gene copies within a range of approximately 1,000 to 10,000 DNA gene copies per liter.

In one embodiment, simultaneous detection of the mcyE/mcyA, sxtA, cyrA, or anaC genes indicates possible production of microcystin, saxitoxin, cylindrospermopsin or anatoxin, respectively. In this exemplary embodiment, the RT-qPCR/qPCR assay detects the presence of cyanotoxin genes in control samples and collected water samples or other test samples. In various embodiments, CTP Assay 100 can be used to determine the total number of gene copies for each cyanotoxin gene and estimate the population size of each group of toxic cyanobacteria.

In the exemplary embodiment shown, each primer pair selected for qPCR analysis targets a sequence of cyanotoxin biosynthesis genes and genus-specific genes that is common to multiple cyanobacteria species. The target genes encode cyanotoxins, including microcystin, anatoxin, saxitoxin, and cylindrospermopsin. Targeted genes include an mcyA gene sequence carried by cyanobacteria in all six genera, an anaC gene sequence carried by cyanobacteria in the Anabaena and Aphanizomenon genera (exemplary detected species include Aphanizomenon gracile, Anabaena sp., and Anabaena circinalis), an sxtA gene sequence carried by cyanobacteria in the Anabaenaand Aphanizomenon genera, and a cyrA gene sequence carried by cyanobacteria in the Anabaena, Aphanizomenon, Cylindrospermopsis, and Raphidiopsis genera (exemplary detected species include Raphidiopsis curvata and Cylindrospermopsis raciborskii).

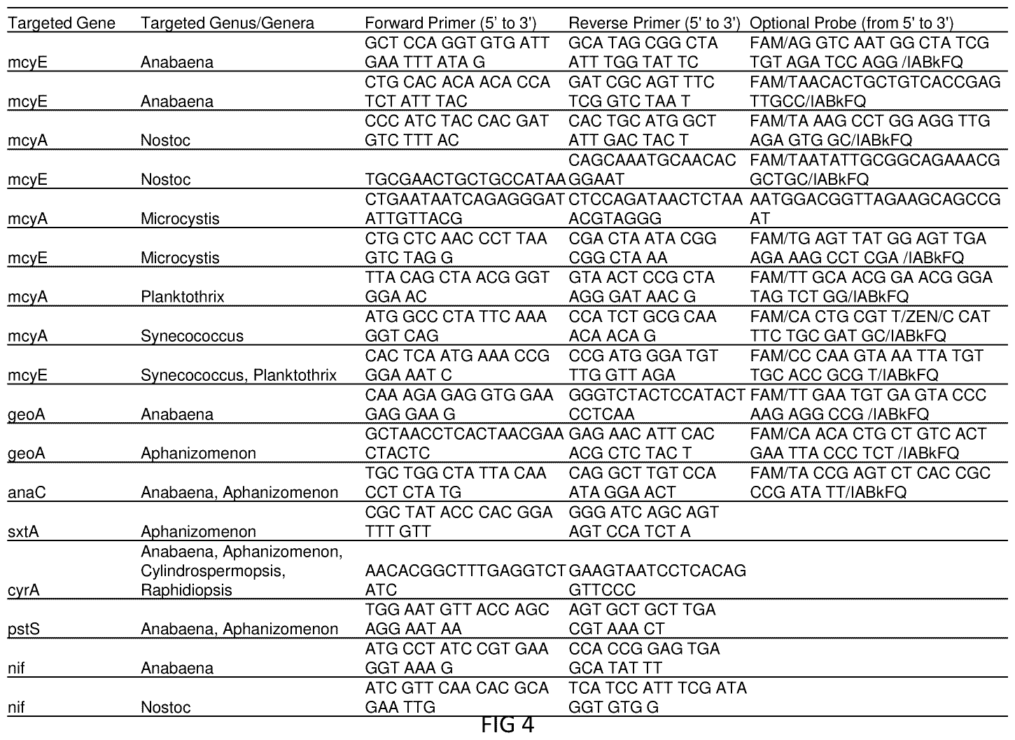

FIG. 4 is a table illustrating primer and probe sequences which can be used to produce CTP Assay Panel 200 for identifying dominant toxic groups of cyanobacteria.

CTP Assay Panel 200 can detect multiple toxic species simultaneously. In various embodiments, CTP Assay Panel 200 can detect the number of toxic gene copies and predict the level of toxin that will be produced by each type of cyanobacteria individually and in the aggregate.

In an alternative embodiment, CTP Assay Panel 200 is comprised of a panel of multiple RT-qPCR/qPCR assays that include the primers shown in FIG. 4A. In this embodiment, the RT-qPCR/qPCR assay detects the presence or absence of individual toxin-producing subtypes of cyanobacteria to determine dominant toxic groups in control samples and collected water samples or other test samples. This embodiment more specifically determines which individual subtypes of cyanobacteria are present and which has the highest population. Each primer shown has an annealing temperature of approximately 60 to 64 degrees Celsius. In various embodiments, the recommended annealing temperature for the primers shown is 62 degrees Celsius.

In the alternative embodiment, alternative primer pairs can detect an mcyA or mcyE gene sequence carried by cyanobacteria in the Anabaena, Nostoc, Microcystis, Planktothrix, and Synecococcus genera (exemplary detected species include Anabaena sp., Anabaenopsis elenkinii, Anabaena lemmermannii, Anabaena flos-aquae, Nostoc sp., Fischerella sp., Nodularia spumigena, Nodularia sphaerocarpa, Nodularia sp., Microcystis sp., M. aeruginosa, M. viridis, M. panniformis, M. wesenbergii, M. smithii, Planktothrix sp., P. rubescens, P. agardhii, Synechococcus sp., WH 8103, and WH8102), an anaC gene sequence carried by cyanobacteria in the Anabaena, and Aphanizomenon genera, an sxtA gene sequence carried by cyanobacteria in the Aphanizomenon genus, a cyrA gene sequence carried by cyanobacteria in the Anabaena, Aphanizomenon, Cylindrospermopsis, and Raphidiopsis genera (exemplary detected species include Raphidiopsis curvata and Cylindrospermopsis raciborskii), a geoA gene sequence carried by cyanobacteria in the Anabaena and Aphanizomenon genera (exemplary detected species include Dolichospermum ucrainicum, D. planctonicum, D. circinale, Nicotiana attenuate, and Anabaena ucrainica), a pstS phosphase gene sequence carried by cyanobacteria in the Anabaena and Aphanizomenon genera, and a nif gene sequence carried by cyanobacteria in the Anabaena and Nostoc genera.

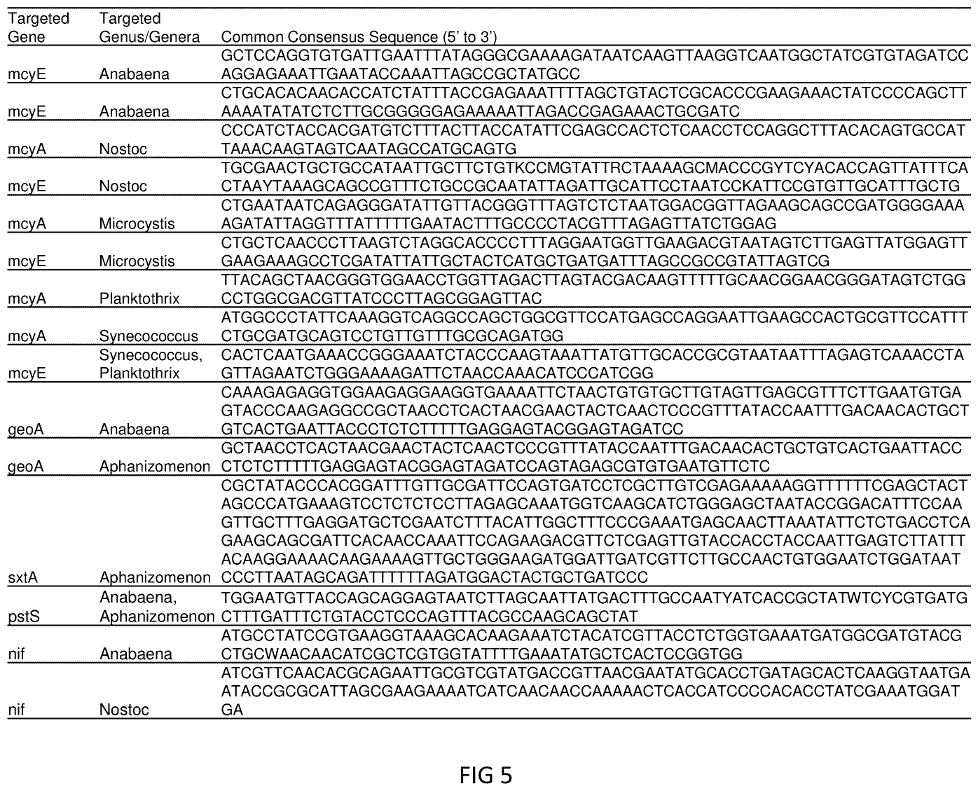

FIG. 5 illustrates the common genetic sequences which are detected by the primers and probes used to produce CTP Assay Panel 200 for identifying dominant toxic groups of cyanobacteria.

FIG. 5 illustrates the common DNA sequences which define a member of the cyanobacteria carrier group detected by the CTP Assay Panel 200 primers and probes.

The exemplary common microcystin sequences shown in FIG. 2 and FIG. 5 are listed as <SEQ ID NO. 52> through <SEQ ID NO. 61> in the sequence listing file. Exemplary common anatoxin sequences are listed as <SEQ ID NO. 64>. Exemplary common saxitoxin sequences are listed as <SEQ ID NO. 65> and <SEQ ID NO. 66>. Exemplary common cylindrospermopsin sequences are listed as <SEQ ID NO. 67>. Exemplary common geoA sequences are listed as <SEQ ID NO. 62> and <SEQ ID NO. 63>.

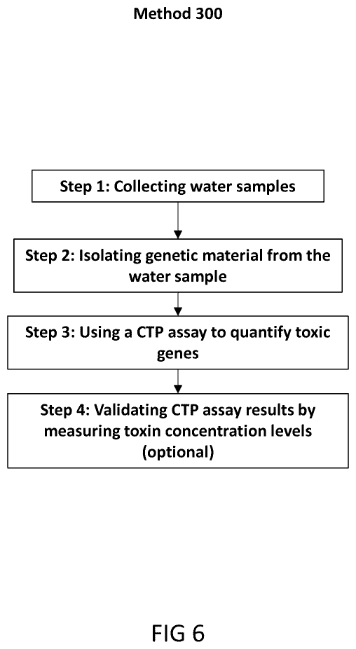

FIG. 6 illustrates exemplary Method 300 for using CTP Assay Panel 100 and/or 200 to measure cyanotoxin genes and correlating CTP Assay results to subsequently measured cyanotoxin levels.

In the exemplary embodiment shown, Method 300 utilizes a panel of novel qPCR/RT-qPCR assays for simultaneously detecting microcystin, anatoxin, saxitoxin, and cylindrospermopsin genes in cyanobacteria. The invention is a testing method for detecting specific bacterial groups associated with toxin production.

In various embodiments, Method 300 may be used to identify the number of gene copies present and predict the amount of toxin that will be produced by each cyanobacteria genus individually and in the aggregate. In various embodiments, Method 300 utilizes analysis of the qPCR/RT-qPCR results to predict whether cyanotoxin concentrations in a source of water will be exceed a toxic threshold deemed harmful to humans and other species within a specified period of time. In various embodiments, the toxic threshold is a limit set by U.S. EPA Drinking Water Health Advisories. For example, the threshold for combined microcystin toxins is 0.3 μg/liter and a gene copy number of 1,000 to 10,000 DNA gene copies per liter predicts that the toxic threshold will be exceeded seven days after measuring the gene copy number.

Step 1 is the step of collecting water samples. In various embodiments, this step is accomplished by periodically collecting water samples from the same source, at various points in time.

Step 2 is the step of isolating genetic material from a water sample.

In one exemplary embodiment this step is accomplished by dividing samples 100-300 mL aliquots and individually filtering the aliquots using EMD Millipore DuraporeTM membrane filters (0.40 μm, MilliPore, Foster City, CA) for DNA extraction. In one embodiment, DNA and RNA are extracted using a kit known in the art, such as AllPrep DNA (QIAGEN, Valencia, CA). Filtered aliquots are stored at −80° C.in 1.5 mL microtubes with lysis buffer prior to extracting DNA and RNA.

In various embodiments, this step includes using any method known in the art for isolating or extracting genetic material from a water sample and conducting reverse transcription to create template DNA from RNA.

Step 3 is the step of using CTP Assay Panel 100 and/or 200 to determine the number of copies of toxic genes.

To conduct a qPCR/RT-qPCR assay, components are combined and heated to create a polymerase chain reaction. In one exemplary embodiment, each reaction contains 1 μM concentration of each selected primer, 2 μl of template DNA from either the sample or the control, a 0.2 mM concentration of each of the four deoxynucleoside triphosphates (dTTP, dCTP, dGTP, and dATP), 1.5 mM MgCl2, 1 μM (each) primer, and 2.5 U of TaqDNA polymerase (Clone Tech, Mountain View, CA) in a total volume of 25 μl. In various embodiments, the effective primer concentration range for the PCR reaction is approximately 0.5 to 1 μM. In this embodiment, the reactions are heated and cooled during 25 cycles of temperature changes, wherein each cycle includes 1 minute of denaturation at 94° C., 1 minute of primer annealing at 62° C., and 5 minutes of primer extension at 72° C. In various embodiments, the annealing temperature is approximately 60 to 64° C.

In various embodiments, this step further includes analyzing the results by methods known in the art to determine the gene copy number (X_{gene}) in each sample, for each cyanotoxin gene detected in that sample. In various embodiments, this step may include running CTP Assay Panel 100 and/or 200 on a Juno robot platform where 40 assays can be run at one time, including 1,600 reactions.

Step 4 is the optional step of validating CTP Assay Panel 100 and/or 200 results by measuring toxin concentration levels on a subsequent date using a testing method known in the art and comparing the measured toxin concentration levels to the results of CTP Assay Panel 100.

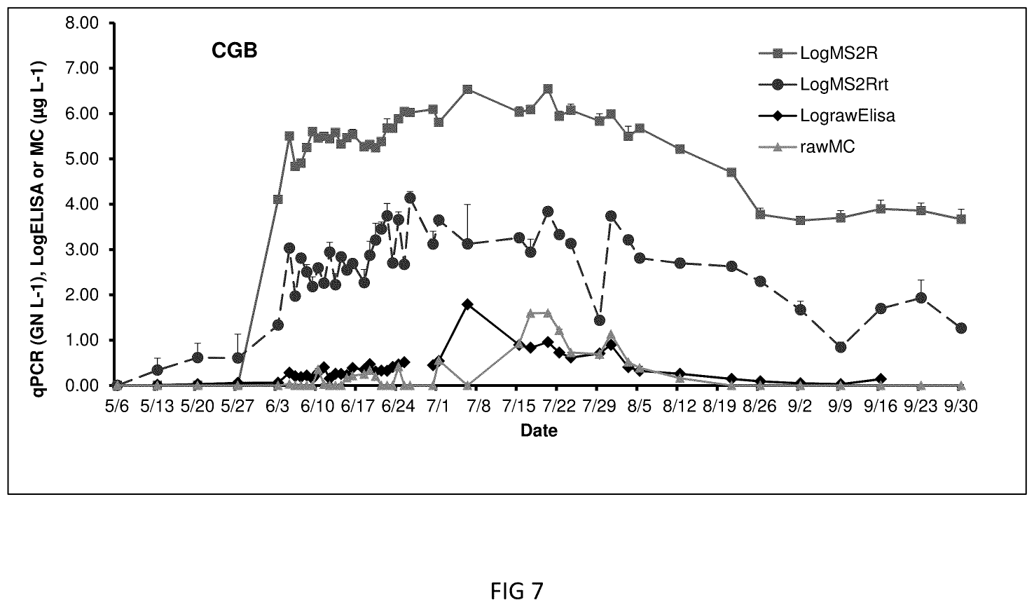

FIG. 7 summarizes exemplary data reporting the number of toxic gene copies and the concentration of cyanotoxins measured during periodic testing of a water source. FIG. 7 summarizes the number of toxic gene copies in DNA isolated from a water source and measured once per week between May 6 and September 30. Data marked LogMS2R were measured by a quantitative polymerase chain reaction (qPCR) assay (represented by squares) and data marked LogMS2Rrt were measured by a reverse transcription quantitative polymerase chain reaction (RT-qPCR) assay (represented by circles).

In the exemplary embodiment shown, the concentration of cyanotoxins in a water source was measured by an enzyme-linked immunosorbent assay (ELISA), represented by diamonds. The raw concentration of cyanotoxins measured by ELISA is represented by triangles.

The x-axis shows dates and the y-axis shows gene copy number or toxin concentration on a logarithmic scale.

In alternative embodiments, the concentration of cyanotoxins in a water source is measured by liquid chromatography-tandem mass spectrometry (LC-MS/MS).

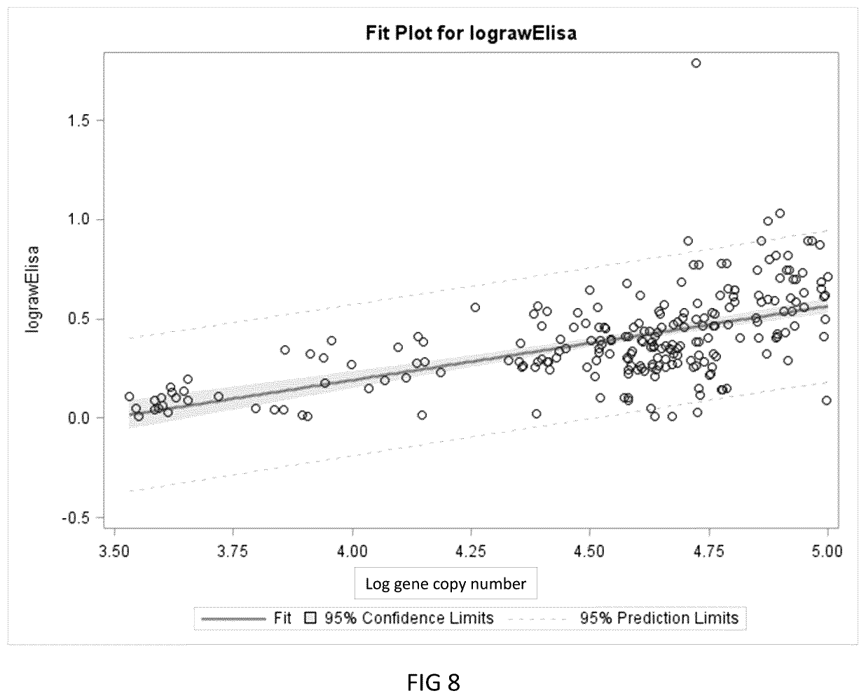

FIG. 8 summarizes exemplary data correlating the number of toxic gene copies measured by CTP Assay Panel 100 and/or 200 to the subsequent concentration of cyanotoxins measured by an enzyme-linked immunosorbent assay (ELISA).

FIG. 8 shows a regression, which is the best fit curve, of the correlation between the number of toxic gene copies measured by qPCR and RT-qPCR and the concentration of cyanotoxins in a water source measured by an enzyme-linked immunosorbent assay (ELISA). FIG. 8 demonstrates that there is a direct correlation between the expression of the toxic gene and the production of the toxin. The gene copy number is plotted on the x-axis on a logarithmic scale and the associated cyanotoxin concentration measured seven days after the gene copy number from the same water source is plotted on the y-axis on a logarithmic scale. In the exemplary embodiment shown, measured data are represented by open circles. The best fit curve showing a predicted toxin concentration on the y-axis for a given gene copy number measured by CTP Assay Panel 100 or 200 is represented by a dark, thin solid line. The 95% confidence interval of the best fit curve is represented by a thicker, lighter line. The 95% prediction limits showing a range of toxin concentration levels predicted by a given gene copy number are represented as dashed lines.

What is claimed is:

1. A qPCR/RT-qPCR assay panel apparatus for detecting the presence of toxic cyanobacterial genes which may be present in fresh water supplies, wherein said assay apparatus is comprised of:
a first qPCR assay for detecting a microcystin gene having a common microcystin sequence;
a second qPCR assay for detecting an anatoxin gene having a common anatoxin sequence;
a third qPCR assay for detecting a saxitoxin gene having a common saxitoxin sequence; and
a fourth qPCR assay for detecting a cylindrospermopsin gene having a common cylindrospermopsin sequence, wherein said first, second, third and fourth assay are effective under standardized testing conditions.

2. The apparatus of claim 1 wherein said standardized testing conditions are selected from a group consisting of annealing temperature, thermocycle duration, and control samples.

3. The apparatus of claim 2, wherein said annealing temperature is approximately 60 to 64 degrees Celsius.

4. The apparatus of claim 2, wherein said annealing temperature is approximately 62 degrees Celsius.

5. The apparatus of claim 1, wherein said first qPCR assay includes at least one primer pair for detecting said microcystin gene, which is selected from a group consisting of:
forward primer 5′-AAAAGTGTTTTATTAGCGGCTCATT-3′ (mcyA_All_F) <SEQ ID NO. 1> with reverse primer 5′-TCTAACCGTCCATTAGAGACTAAACC-3′ (mcyA_All_R) <SEQ ID NO. 2>;
forward primer 5′-CTGCACACAACACCATCTATTTAC-3′ (mcyE_Ana_F1) <SEQ ID NO. 3> and reverse primer 5′-GATCGCAGTTTCTCGGTCTAAT-3′ (mcyE_Ana_R1) <SEQ ID NO. 4>;
forward primer 5′-GCTCCAGGTGTGATTGAATTTATAG-3′ (mcyE_Ana_F3) <SEQ ID NO. 5> and reverse primer 5′-GCATAGCGGCTAATTTGGTATTC-3′ (mcyE_Ana3_R3) <SEQ ID NO. 6>;
forward primer 5′-CCCATCTACCACGATGTCTTTAC-3′ (mcyA_Nost_F) <SEQ ID NO. 7> and reverse primer 5′-CACTGCATGGCTATTGACTACT-3′ (mcyA_Nost_R) <SEQ ID NO. 8>;
forward primer 5′-TGCGAACTGCTGCCATAA-3′ (mcyE_Nost_F) <SEQ ID NO. 9> and reverse primer 5′-CAGCAAATGCAACACGGAAT-3′ (mcyE_Nost_R) <SEQ ID NO. 10>;
forward primer 5′-CTGAATAATCAGAGGGATATTGTTACG-3′ (mcyA_Mic_F) <SEQ ID NO. 11> and reverse primer 5′-CTCCAGATAACTCTAAACGTAGGG-3′ (mcyA_Mic_R) <SEQ ID NO. 12>;
forward primer 5′-CTGCTCAACCCTTAAGTCTAGG-3′ (mcyE_Mic_F4) <SEQ ID NO. 13> and reverse primer 5′-CGACTAATACGGCGGCTAAA-3′ (mcyE_Mic_R4) <SEQ ID NO. 14>;
forward primer 5′-TTACAGCTAACGGGTGGAAC-3′ (mcyA_Pla_F) <SEQ ID NO. 15> and reverse primer 5′-GTAACTCCGCTAAGGGATAACG-3′ (mcyA_Pla_R) <SEQ ID NO. 16>;
forward primer 5′-ATGGCCCTATTCAAAGGTCAG-3′ (mcyA_Syn_F4) <SEQ ID NO. 17> and reverse primer 5′-CCATCTGCGCAAACAACAG-3′ (mcyA_Syn_R4) <SEQ ID NO. 18>; and
forward primer 5′-CACTCAATGAAACCGGGAAATC-3′ (mcyE_Pla_Syn_F1) <SEQ ID NO. 19> and reverse primer 5′-CCGATGGGATGTTTGGTTAGA-3′ (mcyE_Pla_Syn_R1) <SEQ ID NO. 20>.

6. The apparatus of claim 1, wherein said second qPCR assay includes at least one primer pair for detecting said anatoxin gene, which is selected from a group consisting of:
forward primer 5′-TGCTGGCTATTACAACCTCTATG-3′ (anaC_AphAna_F4) <SEQ ID NO. 21> and reverse primer 5′-CAGGCTTGTCCAATAGGAACT-3′ (anaC_AphAna_R4) <SEQ ID NO. 22>.

7. The apparatus of claim 1, wherein said third qPCR assay includes at least one primer pair for detecting said saxitoxin gene, which is selected from a group consisting of:
forward primer 5′-GCGGGACTTTATGCTCTACTAC-3′ (sxtA_AphAna_F1) <SEQ ID NO. 23> and reverse primer 5′-TACTCCGTCATCGGCATTTG-3′ (sxtA_AphAna_R1) <SEQ ID NO. 24>; and
forward primer 5′-CGCTATACCCACGGATTTGTT-3′ (sxtA_Aph_F1) <SEQ ID NO. 25> and reverse primer 5′-GGGATCAGCAGTAGTCCATCTA-3′ (sxtA_Aph_R1) <SEQ ID NO. 26>.

8. The apparatus of claim 1, wherein said fourth qPCR assay includes at least one primer pair for detecting said cylindrospermopsin gene, which is selected from a group consisting of:
forward primer 5′-AACACGGCTTTGAGGTCTATC-3′ (cyrAf_F7) <SEQ ID NO. 27> and reverse primer 5′-GAAGTAATCCTCACAGGTTCCC-3′ (cyrAf_R7) <SEQ ID NO. 28>.

9. The apparatus of claim 1, wherein each of said first qPCR assay, said second qPCR assay, said third qPCR assay and said fourth qPCR assay is comprised of primers that each achieve a primer concentration of approximately 1 μM.

10. A system for detecting the presence of toxic cyanobacterial genes which may be present in fresh water supplies, comprised of:
a first qPCR/RT-qPCR assay panel apparatus for detecting the presence of one or more cyanotoxin genes selected from the group consisting of microcystin, saxitoxin, anatoxin, and cylindrospermopsin; and
a second qPCR/RT-qPCR assay panel apparatus for detecting the presence of said one or more cyanotoxin genes selected from the group consisting of microcystin, saxitoxin, anatoxin, and cylindrospermopsin, wherein said one or more cyanotoxin genes is produced by bacteria selected from the group consisting of Microcystis, Nostoc, Planktothrix, Synecococcus, Anabaena, Aphanizomenon, Raphidiopsis, Cylindrospermopsis, Anabaena sp., Anabaenopsis elenkinii, Anabaena lemmermannii, Anabaena flosaquae, Nostoc sp., Fischerella sp., Nodularia spumigena, Nodularia sphaerocarpa, Nodularia sp., Microcystis sp., M. aeruginosa, M. viridis, M. panniformis, M. wesenbergii, M. smithii, Planktothrix sp., P. rubescens, P. agardhii, Synechococcus sp., Raphidiopsis curvata, Cylindrospermopsis raciborskii, Dolichospermum ucrainicum, D. planctonicum, D. circinale, Nicotiana attenuate, Anabaena ucrainica, Aphanizomenon gracile, and Anabaena circinalis.

11. A method for using a qPCR/RT-qPCR assay panel apparatus for detecting the presence of toxic cyanobacterial genes which may be present in fresh water supplies, comprised of the steps of simultaneously:
performing a first qPCR assay for detecting a microcystin gene having a common microcystin sequence under standardized testing conditions;
performing a second qPCR assay for detecting an anatoxin gene having a common anatoxin sequence under standardized testing conditions;
performing a third qPCR assay for detecting a saxitoxin gene having a common saxitoxin sequence under standardized testing conditions; and
performing a fourth qPCR assay for detecting a cylindrospermopsin gene having a common cylindrospermopsin sequence under standardized testing conditions.

12. The method of claim 11 which further includes the step of selecting said standardized testing conditions from a group consisting of annealing temperature, thermocycle duration, and control samples.

13. The method of claim 11, which further includes the step of selecting an annealing temperature of approximately 60 to 64 degrees Celsius as a standardized testing condition.

14. The method of claim 11, which further includes the step of selecting an annealing temperature of approximately 62 degrees Celsius as a standardized testing condition.

15. The method of claim 11, which further includes the step of selecting at least one primer pair for detecting a microcystin gene from a group consisting of:
forward primer 5′-AAAAGTGTTTTATTAGCGGCTCATT-3′ (mcyA_All_F) <SEQ ID NO. 1> and reverse primer 5′-TCTAACCGTCCATTAGAGACTAAACC-3′ (mcyA_All_R) <SEQ ID NO. 2>;
forward primer 5′-CTGCACACAACACCATCTATTTAC-3′ (mcyE_Ana_F1) <SEQ ID NO. 3> and reverse primer 5′-GATCGCAGTTTCTCGGTCTAAT-3′ (mcyE_Ana_R1) <SEQ ID NO. 4>;
forward primer 5′-GCTCCAGGTGTGATTGAATTTATAG-3′ (mcyE_Ana_F3) <SEQ ID NO. 5> and reverse primer 5′-GCATAGCGGCTAATTTGGTATTC-3′ (mcyE_Ana3_R3) <SEQ ID NO. 6>;
forward primer 5′-CCCATCTACCACGATGTCTTTAC-3′ (mcyA_Nost_F) <SEQ ID NO. 7> and reverse primer 5′-CACTGCATGGCTATTGACTACT-3′ (mcyA_Nost_R) <SEQ ID NO. 8>;
forward primer 5′-TGCGAACTGCTGCCATAA-3′ (mcyE_Nost_F) <SEQ ID NO. 9> and reverse primer 5′-CAGCAAATGCAACACGGAAT-3′ (mcyE_Nost_R) <SEQ ID NO. 10>;
forward primer 5′-CTGAATAATCAGAGGGATATTGTTACG-3′ (mcyA_Mic_F) <SEQ ID NO. 11> and reverse primer 5′-CTCCAGATAACTCTAAACGTAGGG-3′ (mcyA_Mic_R) <SEQ ID NO. 12>;
forward primer 5′-CTGCTCAACCCTTAAGTCTAGG-3′ (mcyE_Mic_F4) <SEQ ID NO. 13> and reverse primer 5′-CGACTAATACGGCGGCTAAA-3′ (mcyE_Mic_R4) <SEQ ID NO. 14>;
forward primer 5′-TTACAGCTAACGGGTGGAAC-3′ (mcyA_Pla_F) <SEQ ID NO. 15> and reverse primer 5′-GTAACTCCGCTAAGGGATAACG-3′ (mcyA_Pla_R) <SEQ ID NO. 16>;
forward primer 5′-ATGGCCCTATTCAAAGGTCAG-3′ (mcyA_Syn_F4) <SEQ ID NO. 17> and reverse primer 5′-CCATCTGCGCAAACAACAG-3′ (mcyA_Syn_R4) <SEQ ID NO. 18>; and
forward primer 5′-CACTCAATGAAACCGGGAAATC-3′ (mcyE_Pla_Syn_F1) <SEQ ID NO. 19> and reverse primer 5′-CCGATGGGATGTTTGGTTAGA-3′ (mcyE_Pla_Syn_R1) <SEQ ID NO. 20>.

16. The method of claim 11 which further includes the step of selecting at least one primer pair for detecting an anatoxin gene from a group consisting of:
forward primer 5′-TGCTGGCTATTACAACCTCTATG-3′ (anaC_AphAna_F4) <SEQ ID NO. 21> and reverse primer 5′-CAGGCTTGTCCAATAGGAACT-3′ (anaC_AphAna_R4) <SEQ ID NO. 22>.

17. The method of claim 11, which further includes the step of selecting at least one primer pair for detecting a saxitoxin gene from a group consisting of:
forward primer 5′-GCGGGACTTTATGCTCTACTAC-3′ (sxtA_AphAna_F1) <SEQ ID NO. 23> and reverse primer 5′-TACTCCGTCATCGGCATTTG-3′ (sxtA_AphAna_R1) <SEQ ID NO. 24>; and
forward primer 5′-CGCTATACCCACGGATTTGTT-3′ (sxtA_Aph_F1) <SEQ ID NO. 25> and reverse primer 5′-GGGATCAGCAGTAGTCCATCTA-3′ (sxtA_Aph_R1) <SEQ ID NO. 26>.

18. The method of claim 11, which further includes the step of selecting at least one primer pair for detecting a cylindrospermopsin gene from a group consisting of:
forward primer 5′-AACACGGCTTTGAGGTCTATC-3′ (cyrAf_F7) <SEQ ID NO. 27> and reverse primer 5′-GAAGTAATCCTCACAGGTTCCC-3′ (cyrAf_R7) <SEQ ID NO. 28>.

19. The method of claim 11, which further includes the step of achieving a primer concentration of approximately 1 μM.

20. The method of claim 11, which further includes the step of obtaining one or more values reflecting the number of copies, X_{gene}, of at least one cyanotoxin gene present in at least one of said fresh water supplies, wherein said cyanotoxin gene is selected from a group consisting of microcystin, anatoxin, saxitoxin, and cylindrospermopsin.
